(19) United States
(12) Patent Application Publication
(10) Pub. No.: US 2002/0115649 A1
(43) Pub. Date: Aug. 22, 2002

(54) Use of texaphyrins in macrophage-mediated disease

(75) Inventors: Kathryn W. Woodburn, Sunnyvale, CA (US); Stuart W. Young, Portola Valley, CA (US)

Correspondence Address: Gloria L. Norberg, Patent Agent, Akin, Gump, Strauss, Hauer & Feld, L.L.P., 1900 Frost Bank Plaza, 816 Congress Avenue, Austin, TX, 78701, US

(73) Assignee: Pharmacyclics, Inc.

(21) Appl. No.: 09/755,795

(22) Filed: Jan. 5, 2001

Related U.S. Application Data
(63) Continuation of application No. PCT/US99/15199, filed on Jul. 6, 1999.
(60) Provisional application No. 60/155,208, filed on Jul. 6, 1998.

(51) Int. Cl. A61B 5/055; A61K 31/555; A61K 31/409

(52) U.S. Cl. 514/185; 514/410; 604/20

(57) ABSTRACT

The present invention provides methods for treating disease of a subject in need thereof by sensitizing the effects of a co-therapeutic agent in macrophages. The method comprises administering a texaphyrin and a co-therapeutic agent to the subject. Texaphyrins are provided for enhancing the cytotoxicity of therapeutic agents in macrophage-mediated disease since texaphyrins have been shown to accumulate in macrophage.

### BACKGROUND OF THE INVENTION

Cardiovascular disease entails the presence of undesirably proliferating cells in the heart or blood vessels, including arteriosclerosis and atherosclerosis. Arteriosclerosis results in degenerative changes and fibrosis in small arteries (arterioles), while atherosclerosis is a disease of medium- and large-sized muscular and elastic arteries such as the coronary arteries, the aorta, the carotid, major arteries supplying the brain, and arteries supplying the peripheral vasculature, particularly, the leg arteries, such as the iliac and femoral arteries.

Over time, atherosclerosis causes significant narrowing through a build-up of lesions (or “plaque”) in one or more arteries. In the peripheral vasculature, this can lead to gangrene and loss of function of the extremities. When coronary arteries narrow more than 50-70%, the blood supply beyond the plaque becomes inadequate, e.g., to meet the increased oxygen demand during exercise. Lack of oxygen (or ischemia) in the heart muscle usually causes chest pain (or “angina”) in most patients. However, 25% of patients experience no chest pain at all despite documented ischemia; these patients have “silent angina” and have the same risk of heart attack as those with angina. When arteries are narrowed in excess of 90-99%, patients often have angina even when at rest. In those cases where a blood clot forms on the plaque, the artery can become completely blocked, causing death of the associated heart muscles.

Attempts to treat atheroma include efforts designed to lower plasma cholesterol levels. When atheromas are symptomatic, vascular interventions such as angioplasty, atherectomy, endarterectomy, coronary artery bypass graft, and internal mammary bypass are considered. Angioplasty (also termed percutaneous transluminal coronary angioplasty (PTCA)), coronary artery balloon dilation, or balloon angioplasty have been used to enlarge narrowed arteries. In this procedure, a catheter with a deflated balloon on its tip is passed into the narrowed part of the artery. The balloon is then inflated, and the narrowed area widened.

Laser angioplasty is a further technique used to open coronary arteries blocked by plaque. A catheter conducting laser light to its tip is inserted into an artery and advanced through the blood vessels to the blocked coronary artery. The laser emits pulsating beams of light that vaporize the plaque. This procedure has been used alone and in conjunction with balloon angioplasty.

Atherectomy is another procedure for opening coronary arteries blocked by plaque. Coronary atherectomy uses a rotating shaver, which is a burr-like device on the end of a catheter. The catheter is introduced into the body through a blood vessel in the leg or arm and is threaded through the blood vessels into the blocked coronary artery. The tip of the catheter has a high-speed rotating device that grinds the plaque up into minute particles. One such device is called a rotablator, which rotates at close to 200,000 rpm, grinding away fatty material that blocks arteries. Balloon angioplasty may then be used on the area treated by atherectomy. Further advances in catheter technology include low pressure balloon angioplasty, rotoblator, atherectomy catheters, hot and cold laser catheters, transluminal extraction catheters (TEC), and ultrasonic ablation.

“Restenosis” is a re-narrowing or re-constricting due to induction of neointimal hyperplasia following vascular intervention. As many as 50 percent of arteries treated by angioplasty and 30 to 40 percent of coronary arteries that undergo bypass restenose, either at the site of a preexisting lesion after angioplasty or at the perianastomotic sites of a bypass, where a normal artery or vein is tied into a blocked coronary artery. The restenosed tissue contains both migratory and proliferative smooth muscle cells as well as macrophage infiltrates (characteristic of wound repair) at the site of trauma induced by the surgical intervention.

Restenosis can occur after interventions including bypass surgery, stenting, vascular grafting, or with dialysis patients, for example. About 25% of such patients are required to undergo a repeat angioplasty to increase coronary artery blood flow. Second angioplasty procedures have similar initial and long-term results as the first procedures. Further, 15% of patients who have had angioplasty will require surgery, or a third angioplasty because of restenosis after a first and second angioplasty.

Abciximab (REOPRO™) is a drug that blocks the sticking of platelets onto cells lining artery walls, thereby lowering the risk of blood clots. REOPRO™ can reduce the risk of artery closure after angioplasty by up to 70%. Sticking of platelets to the cell surface of the artery is also believed to be partially responsible for recurrence of artery narrowing over time. It is not yet known whether REOPRO™ can also reduce the rate of artery restenosis. The major side effect of this drug is an increased risk of bleeding due to its anti-platelet effect.

Stents are expandable supports placed inside arteries and are useful in arteries that restenose repeatedly. The stent is collapsed to a small diameter, placed over an angioplasty balloon catheter, and maneuvered into the constricted area. When the balloon is inflated, the stent locks in place and forms a rigid support to hold the artery open. The stent procedure is sometimes used as an alternative to coronary artery bypass surgery. It is usually reserved for lesions that do not respond to angioplasty alone due to reclosure of the expanded artery. Restenosis is also a problem with the stent procedure.

External beam irradiation has been used in animals and humans to try to control the problem of restenosis. This procedure is often not as effective as intravascular irradiation due to difficulty in localizing the dose of irradiation.

A variety of agents have been tried, but have failed to significantly alter post-angioplasty restenosis in human trials including, e.g., antiplatelet agents, anticoagulants, thromboxane antagonists, prostanoids, calcium channel blockers, ace inhibitors, antiproliferative growth factor inhibitors, lipid lowering agents, corticosteroids, and non-steroidal antiinflammatory agents.

An anti-allergic agent, transilast, has been reported as useful in vitro for inhibiting migration and proliferation of, and collagen synthesis by, vascular smooth muscle cells. The same study suggested that use of colchicine and mitomycin C may be limited by systemic side-effects. A clinical trial using chimeric monoclonal antibody FAB fragment C7E3FAB that blocks platelet aggregation has shown a statistically significant clinical improvement. However, there was a significant increase in bleeding complications. Taxol also has been suggested as useful in inhibition of restenosis due to its reported beneficial effects on cellular microtubules. Forms of hyaluronic acid have been reported useful alone and in combination with therapeutic agents to prevent restenosis. Further, anionic sulfated cyclodextrin derivatives have been reported useful in preventing restenosis. There remains, however, a need for therapeutically effective agents and procedures for the treatment of cardiovascular disease, particularly involving restenosis.

U.S. Pat. No. 5,616,114 relates to an apparatus and methods for delivering a controllable and uniform dosage of radiation from a radioactive material to the walls of a blood vessel for preventing restenosis after angioplasty. U.S. Pat. No. 5,556,389 relates to an apparatus and method for treating an occlusion or constriction such as a stenosis in a blood vessel, and an apparatus and method for treating a tumor or cancerous area occurring around a conduit or duct in the body. A radioactive source of material is inserted through the catheter to the site of the stenosis or cancer. U.S. Pat. No. 5,422,362 reportedly provides a method to inhibit the development of intimal hyperplasia following vascular intervention procedures such as angioplasty consisting essentially of administering a green porphyrin, in particular, a hydromonobenzoporphyrin derivative, to the subject concurrent with and following angioplasty. U.S. Pat. No. 5,419,760 relates to administering by means of a vasoabsorbable stent a photosensitizer to maintain the photosensitizer concentration level in atheromatous plaque. U.S. Pat. No. 5,298,018 relates to photodynamic therapy as an adjunctive or stand alone procedure for the treatment of cardiovascular disease. Light timing was found to be critical for inhibition of atheromatic smooth muscle cell proliferation by the photosensitizer. PCT publication WO 94/04147 relates to a process for use of a combination of ionizing radiation in conjunction with certain benzoporphyrin derivative compounds (BPD) to mediate the destruction of diseased or unwanted cells or tissues. BPD necessitates a liposomal or lipophilic formulation, is not rapidly cleared from the body thereby increasing systemic toxicities, produces severe cutaneous photosensitization, and has modest uptake in atheromatous plaque (ratios of 2.8 and 4.1, Photochem. Photobiol. 1997, 65(5) 877-883).

Texaphyrins are aromatic pentadentate macrocyclic “expanded porphyrins” typically complexed with a metal (and sometimes referred to as “metallotexaphyrins”), which have been described as being useful as MRI contrast agents, as radiosensitizers, as chemosensitizers in oncology, and in photodynamic therapy. Texaphyrin is considered as being an aromatic benzannulene containing both 18π- and 22π-electron delocalization pathways. Texaphyrin molecules absorb strongly in the tissue-transparent 650-900 nm range, and they exhibit inherent selective uptake or biolocalization in certain tissues, particularly regions such as, for example, liver, atheroma or tumor tissue. Texaphyrins have exhibited significant tumor selectivity as detected by magnetic resonance imaging (for paramagnetic metal complexes) and by fluorescence. Texaphyrins and water-soluble texaphyrins, method of preparation and various uses have been described in U.S. Pat. Nos. 4,935,498, 5,162,509, 5,252,720, 5,256,399, 5,272,142, 5,292,414, 5,369,101, 5,432,171, 5,439,570, 5,451,576, 5,457,183, 5,475,104, 5,504,205, 5,525,325, 5,559,207, 5,565,552, 5,567,687, 5,569,759, 5,580,543, 5,583,220, 5,587,371, 5,587,463, 5,591,422, 5,594,136, 5,595,726, 5,599,923, 5,599,928, 5,601,802, 5,607,924, 5,622,946, 5,714,328, 5,798,491, 5,776,925 and 5,775,339; PCT publications WO 90/10633, 94/29316, 95/10307, 95/21845, 96/09315, 96/40253, 96/38461, 97/26915, 97/35617, 97/46262, 98/07733, and 98/25648; allowed U.S. patent application Ser. No. 08/975,522; and pending U.S. patent application Ser. Nos. 08/903,099, 08/946,435, 08/975,090, and 08/763,451, converted to a provisional, USSN not yet assigned; each patent, publication, and application is incorporated herein by reference. Gadolinium texaphyrin has been shown to accumulate in the atheromas in human aortas by MRI (U.S. Pat. No. 5,252,720, previously incorporated by reference herein).

An effective method for treating macrophage-mediated diseases (e.g., cardiovascular disease and rheumatoid arthritis) would be advantageous. The present inventors have addressed these problems and provide herein texaphyrins as sensitizers for use with a source of activating energy or a chemotherapeutic agent to treat such disease. Filed on an even date herewith is U.S. patent application Ser. No. 09/111,148, to the use of texaphyrins in sonodynamic therapy, incorporated by reference herein.

### SUMMARY OF THE INVENTION

The present invention relates generally to the use of texaphyrins in the treatment of macrophage-mediated disease, particularly including the treatment of cardiovascular disease such as atheroma, stenosis (including trauma associated with vascular grafts), the prevention of intimal hyperplasia or restenosis, and treatment of inflammatory disease such as rheumatoid arthritis.

The present invention provides methods of treating a macrophage-mediated disease of a subject in need thereof by sensitizing the effects of a co-therapeutic agent in macrophages of the subject. The method comprises administering an effective amount of a texaphyrin and an effective amount of the co-therapeutic agent to the subject. The present invention results from the demonstration, as provided herein, that texaphyrins accumulate in macrophage. Therefore, texaphyrin is capable of selectively treating macrophage-mediated disease, while not harming surrounding normal tissue.

A method of imaging and treating macrophage-mediated disease of a subject in need thereof is a further embodiment of the invention. The method comprises administering an effective amount of a texaphyrin to the subject, imaging the subject, and administering an effective amount of a co-therapeutic agent to the subject.

Another embodiment of the invention is a method for treating cardiovascular disease of a subject in need thereof by sensitizing macrophages of the subject to a co-therapeutic agent. The method comprises administering an effective amount of a texaphyrin to the subject and an effective amount of the co-therapeutic agent to the subject. The method may further comprise performing vascular intervention on the subject and/or placing a stent in the subject. Where the co-therapeutic agent entails the administration of light energy, the macrophage-mediated cardiovascular disease for which treatment is provided in the present invention entails intimal hyperplasia or restenosis.

A stent impregnated with texaphyrin is another embodiment of the invention.

### DETAILED DESCRIPTION OF THE PREFERRED EMBODIMENTS

Definitions and General Parameters

As used in the present specification, the following words and phrases are generally intended to have the meanings as set forth below, except to the extent that the context in which they are used indicates otherwise.

The terms “a” and “an” mean “one or more” when used in this application, including the claims.

“Texaphyrin,” as used herein, means an aromatic pentadentate macrocyclic expanded porphyrin, also described as an aromatic benzannulene containing both 18π- and 22π-electron delocalization pathways. Texaphyrins and water-soluble texaphyrins, method of preparation and various uses have been described in U.S. Pat. Nos. 4,935,498, 5,162,509, 5,252,720, 5,256,399, 5,272,142, 5,292,414, 5,369,101, 5,432,171, 5,439,570, 5,451,576, 5,457,183, 5,475,104, 5,504,205, 5,525,325, 5,559,207, 5,565,552, 5,567,687, 5,569,759, 5,580,543, 5,583,220, 5,587,371, 5,587,463, 5,591,422, 5,594,136, 5,595,726, 5,599,923, 5,599,928, 5,601,802, 5,607,924, 5,622,946, 5,714,328, 5,798,491, 5,776,925 and 5,775,339; PCT publications WO 90/10633, 94/29316, 95/10307, 95/21845, 96/09315, 96/40253, 96/38461, 97/26915, 97/35617, 97/46262, 98/07733, and 98/25648; allowed U.S. patent application Ser. No. 08/975,522; and pending U.S. patent application Ser. Nos. 08/903,099, 08/946,435, 08/975,090, and 08/763,451, converted to a provisional, USSN not yet assigned; each patent, publication, and application is incorporated herein by reference.

“Co-therapeutic agent,” as used herein, means energy or a drug that, when administered in combination with a texaphyrin, provides treatment for a disease in a manner or degree not attained by such energy or drug when administered alone. Co-therapeutic agents include light, radiation, and sonic energies, and chemotherapeutic drugs.

“Atheroma,” as used herein, means atheromatous plaque, i.e., the atherosclerotic collection of cells, cholesterol, lipid material, and other deposits that accumulate in and occlude arteries, especially coronary arteries, including unstable plaque. “Atheroma,” “atherosclerotic plaque,” and “atheromatous plaque” are meant to be equivalent herein.

“Macrophage,” as used herein, means a subendothelially localized cell. In certain aspects of the invention the macrophage is in an oxidatively stressed environment. Activated macrophages, e.g., associated with cardiovascular disease, are reported as having a spongy appearance and may alternatively be referred to as “foam cells”.

“Macrophage-mediated disease,” as used herein, means a disease where undesired macrophage activity occurs resulting in undesired symptoms. A macrophage-mediated disease includes, for example, cardiovascular disease (including intimal hyperplasia, atheroma, and friable or “unstable” plaque), autoimmune disease, (e.g., rheumatoid arthritis, Sjogrens, scleroderma, systemic lupus erythematosus, non-specific vasculitis, Kawasaki's disease, psoriasis, Type I diabetes, pemphigus vulgaris), granulomatous disease (e.g., tuberculosis, sarcoidosis, lymphomatoid granulomatosis, Wegener's granulomatosus), inflammatory disease (e.g., inflammatory lung diseases such as interstitial pneumonitis and asthma, inflammatory bowel disease such as Crohn's disease, and inflammatory arthritis), and in transplant rejection (e.g., in heart/lung transplants).

By “pharmaceutically effective” is meant that dose which will provide treatment for a particular disease state.

“Restenosis,” as used herein, means the development of atheroma, particularly intimal hyperplasia following vascular intervention procedures such as angioplasty. Restenosis results in occlusion of the vasculature and is accompanied by proliferation of cells including smooth muscle cells at the interior of the blood vessels. Restenosis may result from any procedure that traumatizes vascular walls such as femoral-popliteal bypasses, femoral-tibial bypasses, aorto-iliac bypasses, coronary bypasses, percutaneous transluminal angioplasty, balloon angioplasty, laser angioplasty, or atherectomy, for example. Any procedure that involves traumatic manipulation of the vasculature is included in this definition.

“Sensitizing,” as used herein, means that a texaphyrin increases or enhances the cytotoxicity of the co-therapeutic agent compared to the level of cytotoxicity of that agent in the absence of texaphyrin, e.g., a normally noncytotoxic dosage can be cytotoxic to target cells in which texaphyrin has been incorporated. This is described with reference to chemosensitization in U.S. Pat. No. 5,776,925 (incorporated by reference herein). Thus, in the present methods, texaphyrin “sensitizes” the effects of the co-therapeutic agent in macrophages.

The term “treatment” or “treating” means any treatment of a disease in a mammal, including:

(i) preventing the disease, that is, causing the clinical symptoms of the disease not to develop;

(ii) inhibiting the disease, that is, arresting the development of clinical symptoms; and/or

(iii) relieving the disease, that is, causing the regression of clinical symptoms.

Treatment of Macrophage-Mediated Disorders

Lesions of cardiovascular disease begin as a “fatty streak” consisting of intracellular and extracellular deposits of lipid and lipoprotein particles, which are sought by scavenger cells (macrophages) derived from peripheral blood monocytes and variable numbers of lymphocytes. These macrophages can migrate between the endothelium and take up the lipids, giving rise to subendothelially localized cells that develop a spongy appearance in this oxidatively stressed environment and are known as “foam cells.” Believed to participate in a chronic inflammatory process (e.g., phagocytizing the oxidized LDL), these macrophages can also produce certain biologically relevant molecules that induce or inhibit replication of endothelium, smooth muscle, or additional macrophages, and can make chemoattractants for each of these cell types. Such molecules stimulate migration and/or proliferation of smooth muscle cells within the intima or beneath the accumulations of macrophages and T cells in the media.

Macrophages can induce additional monocyte immigration as well as smooth muscle cell migration, and/or proliferation, leading to the enlargement of the fatty streaks, to the formation of intermediate lesions, and to the formation of advanced lesions of fibrous plaques or atheroma. This progression from early grossly detectable disease to atheroma involves cellular processes such as continuing entry of monocytes/macrophages and proliferation of macrophages, smooth muscle cells, and possibly lymphocytes, formation of an extensive fibrous connective tissue matrix by accumulated smooth muscle cells, and accumulation of intracellular and extracellular lipid, especially in the form of free and esterified cholesterol within macrophages and smooth muscle cells in the lesions. As further evidence of the role of macrophage in cardiovascular disease, the antioxidant probucol was reported to diminish the size of atherosclerotic lesions by decreasing the monocyte/macrophage component of the lesions (Ross, R., “Arteriosclerosis, an Overview,” Chapter 2, page 24, in: Molecular Cardiovascular Medicine, ed. E. Haber, Scientific American, Inc. New York, 1995) Thus, the macrophage appears to be a principal inflammatory cellular mediator of the process of cardiovascular disease.

Macrophages are also mediators of other disease states, including autoimmune diseases, granulomatous diseases, inflammatory diseases and transplant (or allograft) rejection. By way of example, rheumatoid arthritis (RA) is a systemic autoimmune disorder involving a chronic, symmetric and erosive synovitis of peripheral joints. It has been suggested that an initial pathologic event in RA may be activation and/or injury of synovial microvascular endothelial cells resulting in swelling and the appearance of gaps between cells. The lumens of these blood vessels are typically occluded with platelet, leukocyte and firbrin thrombi, as well as edema in the subsynovial lining tissue. Mononuclear cells accumulate initially around the abnormal microvasculature beneath the lining cell layer and in the deeper sublining synovial tissues. As the disease progresses, the lining layer can increase from a thickness of 1 to 3 cells to a depth of 5 to 10 cells, composed largely of macrophage-like cells that have been apparently recruited to the joint. This is accompanied by massive tumor-like proliferation and activation of the connective tissue stroma. As in cardiovascular disease, the macrophages in the rheumatic synovium are highly activated, exhibiting a procoagulant activity and serving as a significant source of lipid mediators of inflammation.

Texaphyrins administered with a co-therapeutic agent are known to provide treatment by selectively targeting certain undesirably proliferating lipid-associated cells in cancer and in the photodynamic therapy of atheroma. The present invention results from experimental evidences that texaphyrins accumulate in macrophage, e.g., foam cells associated with atheroma. While the precise mechanisms of action of texaphyrins have yet to be established, and not wishing to be bound by any particular theory, given that atheroma and many macrophage-related disease entail states of oxidative stress, the administration of a combination of texaphyrin and a co-therapeutic agent may exacerbate this fragile equilibrium.

Texaphyrins Employed in the Invention

Exemplary texaphyrins or texaphyrin metal complexes for use in the present invention have structure I or II:

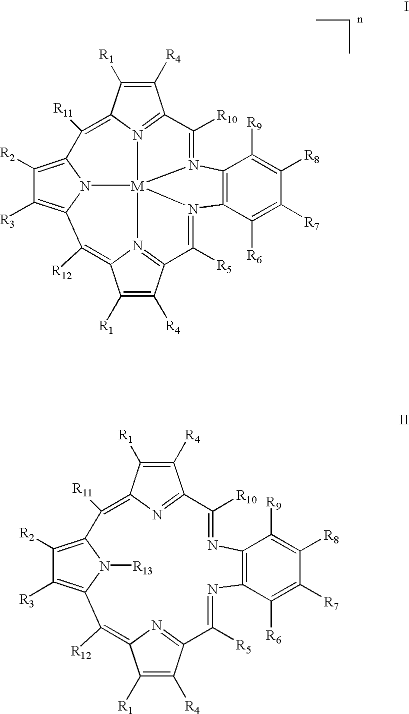

M is H, a divalent metal cation, or a trivalent metal cation. Preferably, M is a divalent metal cation, or a trivalent metal cation. A presently preferred divalent metal cation is Ca(II), Mn(II), Co(II), Ni(II), Zn(II), Cd(II), Hg(II), Fe(II), Sm(II), or UO₂(II). A presently preferred trivalent metal cation is Mn(III), Co(III), Ni(III), Fe(III), Ho(III), Ce(III), Y(III), In(III), Pr(III), Nd(III), Sm(III), Eu(III), Gd(III), Tb(III), Dy(III), Er(III), Tm(III), Yb(III), Lu(III), La(III), or U(III). More preferred trivalent metal cations are presently Lu(III) or Gd(III). The metal cation may be a radioisotope.

Substituents R₁-R₁₃ and “n” are described in U.S. Patents, PCT publications, and allowed and pending patent applications, previuosly incorporated by reference herein. A substituent may be conjugated to a radioisotope.

Presently preferred functionalizations are: when R₆ and R₉ are other than hydrogen, then R₅ and R₁₀ are hydrogen or methyl; and when R₅ and R₁₀ are other than hydrogen, then R₆ and R₉ are hydrogen, hydroxyl, or halide other than iodide. Other preferred functionalizations are where R₆ and R₉ are hydrogen, then R₅, R₁₀, R₁₁ and R₁₂ are independently hydrogen, phenyl, lower alkyl or lower hydroxyalkyl. The lower alkyl is preferably methyl or ethyl, more preferably methyl. The lower hydroxyalkyl is preferably of 1 to 6 carbons and 1 to 4 hydroxy groups, more preferably 3-hydroxypropyl. The phenyl may be substituted or unsubstituted.

More preferred are compounds GdT2BET (M=Gd(III)) and LuT2BET (M=Lu(III)) where the designation T2BET has R groups as follows: R₁ is CH₂(CH₂)₂OH, R₂ and R₃ are CH₂CH₃, R₄is CH₃, R₇ and R₈ are O(CH₂CH₂O)₃CH₃, and R₅, R₆, and R₉-R₁₂ are H. While the cited texaphyrins are presently preferred for use in the present invention, the invention is not limited thereto.

Generally, water soluble texaphyrins retaining lipophilicity are preferred for the applications described herein. “Water soluble” means soluble in aqueous fluids to about 1 mM or better. “Retaining lipophilicity” means having greater affinity for lipid rich tissues or materials than surrounding nonlipid rich tissues. “Lipid rich” means having a greater amount of triglyceride, cholesterol, fatty acids or the like.

Importantly, texaphyrins may be synthesized using certain substituents to effect a lipid-water distribution coefficient that is optimal for use in conjunction with a co-therapeutic agent for methods herein. U.S. Patents, PCT publications, and pending applications to texaphyrins, methods of making and uses thereof have been previously incorporated by reference herein.

Chemosensitization of Macrophage-Mediated Disease

Chemosensitizers are chemical agents or maneuvers traditionally employed in cancer treatment, which are not cytotoxic in themselves, but modify the host, the tumor or a chemotherapeutic agent so as to enhance anticancer therapy. Texaphyrins may be used as chemosensitizers for enhancing the cytotoxicity of a variety of chemotherapeutic agents having differing mechanisms of action and useful for treating disease. For chemosensitization methods of the present invention, the co-therapeutic agent is a chemotherapeutic drug and the target tissue is tissue diseased by macrophage. When administered, chemotherapeutic agents are in contact with plaque, for example, and may accumulate within atheroma or restenotic tissue. Certain currently available chemotherapeutic agents traditionally used in cancer chemotherapy are listed according to class below in Table A and are expected to be effective with texaphyrin in macrophage mediated disease. The listing is not intended to limit the scope of chemotherapeutic agents useful with texaphyrins in the present invention. Bioreductive agents are a subclass of chemotherapeutic agents and are useful with texaphyrins for chemosensitization. 2-Nitroimidazoles and intercalating agents are examples of bioreductive agents. Antioxidants such as probucanol, vitamin E, and L-arginine can also be employed. Hyaluronic acid, a cyclodextrin derivative, an angiotensin converting enzyme (ace) inhibitor such as cilazapril, or colchicine, are further chemotherapeutic agents.

TABLE A
Chemotherapeutic Agents¹

| Class | Type of Agent | Name |
|---|---|---|
| Alkylating Agents | Nitrogen Mustards | Mechlorethamine |
| | | (HN₂) |
| | | Cyclophosphamide |
| | | Ifosfamide |
| | | Melphalan |
| | | Chlorambucil |
| | | Estramustine |
| | Ethylenimines and | Hexamethylmelamine |
| | Methylmelamines | Thiotepa |
| | Alkyl Sulfonates | Busulfan |
| | Nitrosoureas | Carmustine |
| | | Lomustine |
| | | Semustine |
| | | Streptozocin |
| | Triazenes | Dacarbazine |
| | | Procarbazine |
| | | Aziridine |
| Antimetabolites | Folic Acid Analogs | Methotrexate |
| | | Trimetrexate |
| | Pyrimidine Analogs | Fluorouracil |
| | | Floxuridine |
| | | Cytarabine |
| | | Azacitidine |
| | Purine Analogs and | Mercaptopurine |
| | Related Inhibitors | Thioguanine |
| | | Pentostatin |
| | | Fludarabine |
| Natural Products | Vinca Alkaloids | Vinblastine (VLB) |
| | | Vincristine |
| | | Vindesine |
| | Epipodophyllotoxins | Etoposide |
| | | Teniposide |
| | Antibiotics | Dactinomycin |
| | | Daunorubicin |
| | | Doxorubicin |
| | | 4′-Deoxydoxorubicin |
| | | Bleomycin |
| | | Plicamycin |
| | | Mitomycin |
| | Enzymes | L-Asparaginase |
| | Taxanes | Docetaxel |
| | Taxoids | Paclitaxel (Taxol) |
| | Biological Response | Interferon Alfa |
| | Modifiers | Tumor Necrosis Factor |
| | | Tumor-Infiltrating |
| | | Lymphocytes |
| Miscellaneous Agents | Platinum Coordination | Cisplatin |
| | Complexes | Carboplatin |
| | Anthracenedione | Mitoxantrone |
| | Substituted Urea | Hydroxyurea |
| | Methyl Hydrazine | Procarbazine |
| | Derivative | |
| | Adrenocortical | Mitotane |
| | Suppressant | Aminoglutethimide |
| Hormones and | Adrenocorticosteroids | Prednisone |
| Antagonists | | |
| | Progestins | Hydroxyprogesterone |
| | | caproate |
| | | Medroxyprogesterone |
| | | acetate |
| | | Megestrol acetate |
| | Estrogens | Diethylstilbestrol |
| | | Ethinyl estradiol |
| | Antiestrogen | Tamoxifen |
| | Androgens | Testosterone |
| | | propionate |
| | | Fluoxymesterone |
| | Antiandrogen | Flutamide |
| | Gonadotropin- | Leuprolide |
| | releasing hormone | Goserelin |
| | analog | |

¹Adapted from Calabresi, P., and B. A. Chabner, “Chemotherapy of Neoplastic Diseases” Section XII, pp 1202-1263 in: Goodman and Gilman's The Pharmacological Basis of Therapeutics, Eighth ed., 1990 Pergamin Press, Inc.; and Barrows, L. R., “Antineoplastic and Immunoactive Drugs”, Chapter 75, pp 1236-1262, in: Remington: The Science and Practice of Pharmacy, Mack Publishing Co. Easton, PA, 1995.; both references are incorporated by reference herein.

The chemotherapeutic agent administered with a texaphyrin may be one of the following: an alkylating agent such as a nitrogen mustard, an ethylenimine or a methylmelamine, an alkyl sulfonate, a nitrosourea, or a triazene; an antimetabolite such as a folic acid analog, a pyrimidine analog, or a purine analog; a natural product such as a vinca alkaloid, an epipodophyllotoxin, an antibiotic, an enzyme, a taxane, or a biological response modifier; miscellaneous agents such as a platinum coordination complex, an anthracenedione, an anthracycline, a substituted urea, a methyl hydrazine derivative, or an adrenocortical suppressant; or a hormone or an antagonist such as an adrenocorticosteroid, a progestin, an estrogen, an antiestrogen, an androgen, an antiandrogen, or a gonadotropin-releasing hormone analog. Specific examples of alkylating agents, antimetabolites, natural products, miscellaneous agents, hormones and antagonists, and the types of cancer for which these classes of chemotherapeutic agents are indicated are provided in Table A. Preferably, the chemotherapeutic agent is a nitrogen mustard, an epipodophyllotoxin, an antibiotic, an anti-oxidant, or a platinum coordination complex. A more preferred chemotherapeutic agent is bleomycin, doxorubicin, paclitaxel, etoposide, 4-OH cyclophosphamide, cisplatinum, or mitomycin-C. A presently preferred chemotherapeutic agent is doxorubicin, bleomycin, taxol,or mitomycin-C.

The mechanism of action of texaphyrin as a chemosensitizer remains to be established definitively. While not wanting to be bound by theory, it is thought that texaphyrin may inhibit repair of cellular damage caused by the chemotherapeutic agent, texaphyrin may compromise the cell's energy stores, or may increase free radical life span.

The use of texaphyrin as a chemosensitizer has an important advantage due to localization of texaphyrin to macrophage as shown herein. It may thus be possible to administer less chemotherapeutic agent in the presence of texaphyrin to obtain a desired effect. As a result of being exposed to less chemotherapy, the patient may experience less general toxicity, while regions with a concentration of macrophages (such as atheroma, including restenotic tissue and plaque, and the rheumatoid synovium) experience enhanced cytotoxicity.

In one aspect of the invention, a patient having macrophage-mediated cardiovascular disease is administered a dose of texaphyrin at intervals with each dose of the chemotherapeutic agent. A further step of performing angioplasty on the subject, or placing a stent into the subject is a further aspect of the present invention. The stent may be impregnated with the texaphyrin and the chemotherapeutic agent in yet another embodiment of the invention.

Radiation Sensitization of Macrophage-Mediated Disease

Texaphyrin is a known radiosensitizer. Not wanting to be bound by theory, texaphyrin is thought to act as a radiosensitizer in the following manner. Oxygen is the ultimate electron acceptor in physiological systems. However, texaphyrins have a redox potential below that of oxygen. All reductants are expected to reduce texaphyrin, even superoxide, as can be seen from the following listing of redox potentials:

| | | |
|---|---|---|
| e⁻_{aq} = | −2.80 | V. |
| Porphyrin = | −0.6 to −1.8 | V. |
| Quinone = | −0.2 to −1.0 | V. |
| O⁻₂ = | −0.18 | V. |
| GdT2B2* = | +0.08 | V. |

Therefore, gadolinium texaphyrin “soaks up” electrons readily and prevents their reaction with hydroxyl radicals or other oxidized species. This low redox potential of gadolinium texaphyrin is a property of texaphyrin that enhances the amount of radiation damage incurred at the site of the texaphyrin since in the absence of texaphyrin hydroxyl radicals and hydrated electrons recombine and little radiation damage occurs. However, in the presence of texaphyrin hydroxyl radicals are free to cause damage. Furthermore, the trapping of electrons by texaphyrin prevents hydrated electrons from interacting with the hydroxyl radical-induced damage site to repair the damage.

Texaphyrin, therefore, has advantageous properties for use as a radiation sensitizer: 1) The low redox potential of texaphyrin causes hydrated electrons to react with texaphyrin allowing OH to cause damage; 2) The texaphyrin radical is relatively stable, yet reacts readily to modify neighboring molecules covalently; 3) Texaphyrin may be particularly effective for treating hypoxic areas because of intrinsic biolocalization and its indifference to the presence or absence of O₂, it may “replace” oxygen as a redox cycle; 4) Texaphyrin is nontoxic at therapeutic doses; and, as shown herein, 5) Texaphyrin localizes in macrophage, a target cell for inducing macrophage-mediated disease such as cardiovascular disease, autoimmune disease, and the like as described herein.

The radiation sensitization properties of the texaphyrins described herein are expected to allow reduced doses of radiation to be effective in treatment of an individual. Therefore, radiation side effects such as nausea and damage to normal cells may be lessened when treatment includes the use of texaphyrins.

This radiation sensitization property of texaphyrins is related to reduction of the texaphyrin ligand only (i.e. Gd(III) ion is not reduced in this process). In vitro and in vivo studies on the gadolinium texaphyrin complexes demonstrate their potential to enhance radiation damage, and since this enhancement is unaffected by the presence of oxygen, texaphyrins have the potential to increase damage in both oxic and hypoxic areas.

For radiation sensitization methods of the present invention, the co-therapeutic agent is radiation in the form of x-rays, internal or external gamma emitting radioisotopes, or ionizing particles such as α or β particles, for example.

Photodynamic Therapy of Macrophage-Mediated Disease

Photodynamic therapy (PDT) is a treatment technique that uses a photosensitizing dye and non-damaging light corresponding to the sensitizer's absorption profile to produce cytotoxic materials, such as singlet oxygen, from benign precursors when irradiated in the presence of oxygen. Other reactive species such as superoxide, hydroperoxyl, or hydroxyl radicals may be involved in the consequent irreversible damage to biological components. At the doses used, neither the light nor the drug has any independent activity against the target. The effectiveness of PDT is predicated on three additional factors: i) The photosensitive dyes used in PDT preferably have the ability to localize at the treatment site as opposed to surrounding tissue. ii) The high reactivity and short lifetime of activated oxygen means that it has a very short range (˜0.1 ₘm) and is unlikely to escape from the region in which it is produced; cytotoxicity is therefore restricted to the precise region of photoactivated drug. iii) Developments in light delivery, such as lasers, light emitting diodes, and fiber optics, allow a beam of intense, non-damaging, light to be delivered accurately to many parts of the body. For a review of photodynamic therapy, see U.S. Pat. No. 5,252,720 (incorporated by reference herein).

Photosensitive texaphyrins are used for photodynamic therapy. A photosensitive texaphyrin may be a free-base texaphyrin or may be metallated. The term “photosensitive”, as used herein, means that upon photoirradiation by light associated with the absorption profile of texaphyrin, texaphyrin effects the generation of oxygen products that are cytotoxic. Cytotoxic oxygen products may be singlet oxygen, hydroxyl radicals, superoxide, hydroperoxyl radicals, or the like. For generating singlet oxygen, the preferred metal is a diamagnetic metal. Presently, a preferred diamagnetic metal is Lu(III), La(III), In(III), Zn(II), or Cd(II) and a most preferred diamagnetic metal is Lu(III).

For photodynamic therapy methods of the present invention, the co-therapeutic agent is light. After the photosensitizing texaphyrin has been administered, the tissue being treated is irradiated at a wavelength similar to the absorbance of the texaphyrin, usually either about 400-500 nm or about 700-800 nm. In the present photodynamic therapy methods, the light source may be a laser, a light-emitting diode, or filtered light from, for example, a xenon lamp; the light may have a wavelength range of about 400-900 nm, preferably about 400-500 nm or 700-800 nm, more preferably about 450-500 nm or about 710-760 nm, or most preferably about 450-500 nm or about 725-740 nm; and the light may be administered topically, endoscopically, or interstitially (via, e.g., a fiber optic probe). Preferably, the light is administered using a slit-lamp delivery system. A wavelength in this range is especially preferred since blood is relatively transparent at longer wavelengths and, therefore, treatment results in less tissue damage and better light penetration. The fluence and irradiance during the irradiating treatment can vary depending on type of tissue, depth of target tissue, and the amount of overlying fluid or blood.

Sonodynamic Therapy of Macrophage-Mediated Disease

Ultrasound has been used extensively over the last decades for medical diagnosis and physical therapy. Ultrasound has the ability to penetrate deeply into tissues while maintaining the ability to focus energy into small volumes. At high intensities, ultrasound can be focused to penetrate deeply into tissues and cause cell cavitation or cell death. These thermal effects (i.e. cell killing) due to high intensity ultrasound absorption have been widely reported in conjunction with tumor treatment. However, thermogenesis of tumors by ultrasound has not been completely effective as a tumor treatment because the high intensity ultrasound energy causes cell cavitation in the tumors and in surrounding normal tissue, i.e., ultrasound is not selective for tumor cells.

As provided in patent application SN 09/111,148, filed on an even date herewith and incorporated by reference herein, it has been discovered that unlike the porphyrins, which increase the toxicity of a sonodynamic agent only when present in the extracellular matrix (and not when incorporated intracellularly), texaphyrins are suitable intracellular sensitizers. Sonodynamic therapy offers certain advantages over existing radiation and photodynamic therapies. For example, ultrasound can penetrate tissues more effectively than light, facilitating greater access to non-invasive therapy, and can also be focused more effectively as compared to radiation. Diagnostic advantages are also achieved through the use of texaphyrins.

The precise mechanism of action of texaphyrin as an intracellular sensitizer for sonodynamic therapy remains to be definitively established. While not wanting to be bound by any particular theory, it is thought that the texaphyrin may induce cell cavitation, or that formation or prolongation of radical species may occur during sonication, facilitating cell death at sub-lethal sonodynamic agent dosages. Because texaphyrins are capable of sensitizing while intracellularly incorporated and are known to be cleared relatively rapidly from the plasma and extracellular matrix, particularly selective sensitization is achieved.

Administration

The texaphyrin to be used in the methods of the invention will be administered in a pharmaceutically effective amount, employing a method of administration, pharmaceutical formulation, and with a co-therapeutic agent as is known in the art in light of the present disclosure. One of skill in the art in light of the present disclosure would also realize flexibility in the below regimens and would be able to test, without undue experimentation, for optimal timing and dosage for administration of a texaphyrin for a particular circumstance.

Dosages: The specific dose will vary depending on the particular texaphyrin chosen, the dosing regimen to be followed, and the particular co-therapeutic agent with which it is administered, employing dosages within the range of about 0.01 mg/kg/treatment up to about 23 mg/kg/treatment.

Administration for Photodynaminc Therapy: By way of example, lutetium texaphyrin is administered in solution containing 2 mg/ml optionally in 5% mannitol, USP. Dosages of about 1.0 or 2.0 mg/kg to about 4.0 or 5.0 mg/kg, preferably 3.0 mg/kg may be employed, up to a maximum tolerated dose that was determined in one study to be 5.2 mg/kg. The texaphyrin is administered by intravenous injection, followed by a waiting period of from as short a time as several minutes or about 3 hours to as long as about 72 or 96 hours (depending on the treatment being effected) to facilitate intracellular uptake and clearance from the plasma and extracellular matrix prior to the administration of photoirradiation.

Dose levels for certain uses may range from about 0.05 μmol/kg to about 20 μmol/kg administered in single or multiple doses (e.g. before each fraction of light). The lower dosage range would be preferred for intra-arterial injection or for impregnated stents.

The co-administration of a sedative (e.g., benzodiazapenes) and narcotic analgesic are sometimes recommended prior to light treatment along with topical administration of Emla cream (lidocaine, 2.5% and prilocaine, 2.5%) under an occlusive dressing. Other intradermal, subcutaneous and topical anesthetics may also be employed as necessary to reduce discomfort. Subsequent treatments can be provided after approximately 21 days. In certain circumstances involving particular sensitivity to light, the treating physician may advise that certain patients avoid bright light for about one week following treatment.

When employing photodynamic therapy, a target area is treated with light at about 732±16.5 nm (full width half max) delivered by LED device or an equivalent light source (e.g., a Quantum Device Qbeam™ Q BMEDXM-728 Solid State Lighting System, which operates at 728 nm) at an intensity of 75 mW/cm² for a total light dose of 150 J/cm². The light treatment takes approximately 33 minutes.

The optimum length of time following texaphyrin administration until light treatment can vary depending on the mode of administration, the form of administration, and the type of target tissue. Typically, the texaphyrin persists for a period of minutes to hours, depending on the texaphyrin, the formulation, the dose, the infusion rate, as well as the type of tissue and tissue size.

After the photosensitizing texaphyrin has been administered, the tissue being treated is photoirradiated at a wavelength similar to the absorbance of the texaphyrin, usually either about 400-500 nm or about 700-800 nm, more preferably about 450-500 nm or about 710-760 nm, or most preferably about 450-500 nm or about 725-740 mn. The light source may be a laser, a light-emitting diode, or filtered light from, for example, a xenon lamp; and the light may be administered topically, endoscopically, or interstitially (via, e.g., a fiber optic probe). Preferably, the light is administered using a slit-lamp delivery system. The fluence and irradiance during the photoirradiating treatment can vary depending on type of tissue, depth of target tissue, and the amount of overlying fluid or blood. For example, a total light energy of about 100 J/cm² can be delivered at a power of 200 mW to 250 mW depending upon the target tissue.

Administration for Chemosensitization: Texaphyrins may be administered before, at the same time, or after administration of the chemotherapeutic drug. The texaphyrin may be administered as a single dose, or it may be administered as two or more doses separated by an interval of time. The texaphyrin may be administered from about one minute to about 12 hr following administration of the chemotherapeutic drug, preferably from about 5 min to about 5 hr, more preferably about 4 to 5 hr. The dosing protocol may be repeated, from one to three times, for example. A time frame that has been successful in vivo is administration of texaphyrin about 5 min and about 5 hr after administration of a chemotherapeutic agent, with the protocol being performed once per week for three weeks. Administration may be intra-arterial injection, intravenous, intraperitoneal, parenteral, intramuscular, subcutaneous, oral, topical, or via a device such as a stent, for example, with parenteral and intra-arterial administration being preferred, and intra-arterial being more preferred.

Administering a texaphyrin and a chemotherapeutic drug to the subject may be prior to, concurrent with, or following vascular intervention. The method may begin at a time roughly accompanying a vascular intervention, such as an angioplastic procedure, for example. Multiple or single treatments prior to, at the time of, or subsequent to the procedure may be used. “Roughly accompanying a vascular intervention” refers to a time period within the ambit of the effects of the vascular intervention. Typically, an initial dose of texaphyrin and chemotherapeutic drug will be within 6-12 hours of the vascular intervention, preferably within 6 hours thereafter. Follow-up dosages may be made at weekly, biweekly, or monthly intervals. Design of particular protocols depends on the individual subject, the condition of the subject, the design of dosage levels, and the judgment of the attending practitioner.

Administration for Radiation Sensitization: Gadolinium texaphyrin is administered in a solution containing 2 mM optionally in 5% mannitol USP/water (sterile and non-pyrogenic solution). Dosages of 0.1 mg/kg up to as high as about 23.0 mg/kg have been delivered, preferably about 3.0 to about 15.0 mg/kg (for volume of about 90 to 450 mL) may be employed, optionally with pre-medication using anti-emetics above about 6.0 mg/kg. The texaphyrin is administered via intravenous injection over about a 5 to 10 minute period, followed by a waiting period of about 2 to 5 hours to facilitate intracellular uptake and clearance from the plasma and extracellular matrix prior to the administration of radiation.

When employing radiation therapy, a palliative course of 30 Gy in ten (10) fractions of radiation are administered over consecutive days excluding weekends and holidays. In the treatment of brain metastases, whole brain megavolt radiation therapy is delivered with ⁶⁰Co teletherapy or a ≧4 MV linear accelerator with isocenter distances of at least 80 cm, using isocentric techniques, opposed lateral fields and exclusion of the eyes. A minimum dose rate at the midplane in the brain on the central axis is about 0.5 Gy/minute.

Texaphyrins used as radiation sensitizers may be administered before, or at the same time as, or after administration of the ionizing radiation. The texaphyrin may be administered as a single dose, as an infusion, or it may be administered as two or more doses separated by an interval of time. Where the texaphyrin is administered as two or more doses, the time interval between the texaphyrin administrations may be from about one minute to a number of days, preferably from about 5 min to about 1 day, more preferably about 4 to 5 hr. The dosing protocol may be repeated, from one to ten or more times, for example. Dose levels for radiation sensitization may range from about 0.05 μmol/kg to about 20 μmol/kg administered in single or multiple doses (e.g. before each fraction of radiation). A lower dosage range is presently preferred for intra-arterial injection or for impregnated stents. In the case of texaphyrins incorporating or conjugated to a radioisotope, the additional administration of radiation as a co-therapeutic agent is optional.

Administration may be intra-arterial injection, intravenous, intraperitoneal, parenteral, intramuscular, subcutaneous, oral, topical, or via an impregnated or coated device such as a stent, for example, or an artery-inserted cylindrical polymer, with parenteral and intra-arterial administration being preferred, and intra-arterial being more preferred. In one aspect of the invention, a patient having restenosis or at risk for restenosis is administered a dose of texaphyrin at intervals with each dose of radiation.

Administering a texaphyrin to the subject may be prior to, concurrent with, or following vascular intervention, and the intervention is followed by radiation. The method may begin prior to, such as about 24-48 hours prior to, or at a time roughly accompanying vascular intervention, for example. Multiple or single treatments prior to, at the time of, or subsequent to the procedure may be used. “Roughly accompanying the vascular intervention” refers to a time period within the ambit of the effects of the vascular intervention. Typically, an initial dose of texaphyrin and radiation will be within 1-24 hours of the vascular intervention, preferably within about 5-24 hours thereafter. Follow-up dosages may be made at weekly, biweekly, or monthly intervals. Design of particular protocols depends on the individual subject, the condition of the subject, the design of dosage levels, and the judgment of the attending practitioner.

Administration for Sonodynamic Therapy: The use of texaphyrins in sonodynamic therapy is described in U.S. patent application Ser. No. 09/111,148, previously incorporated herein by reference. Texaphyrin is administered before administration of the sonodynamic agent. The texaphyrin may be administered as a single dose, or it may be administered as two or more doses separated by an interval of time. Parenteral administration is typical, including by intravenous and interarterial injection. Other common routes of administration can also be employed.

Ultrasound is generated by a focused array transducer driven by a power amplifier. The transducer can vary in diameter and spherical curvature to allow for variation of the focus of the ultrasonic output. Commercially available therapeutic ultrasound devices may be employed in the practice of the invention. The duration and wave frequency, including the type of wave employed may vary, and the preferred duration of treatment will vary from case to case within the judgment of the treating physician. Both progressive wave mode patterns and standing wave patterns have been successful in producing cavitation of diseased tissue. When using progressive waves, the second harmonic can advantageously be superimposed onto the fundamental wave.

A preferred sonodynamic agent employed in the present invention is ultrasound, particularly is low intensity, non-thermal ultrasound, i.e., ultrasound generated within the wavelengths of about 0.1 MHz and 5.0 MHz and at intensities between about 3.0 and 5.0 W/cm².

Further Administration Protocols: Texaphyrin and a suitable co-therapeutic agent can also be administered in the context of other medical procedures. For example, in allograft transplantation, administration may be accomplished by perfusion of the graft prior to implantation. Following a brief period for uptake, e.g., by macrophages, the remaining texaphyrin is rinsed from the graft followed by application of the co-therapeutic agent. Administration to selectively treat diseases characterized by circulating macroophages may be accomplished, e.g., by extracorporeal contact, filtration of non-absorbed texaphyrin employing a lipophilic filter, followed by application of the co-therapeutic agent.

Pharmaceutical Preparations: Texaphyrins are provided as pharmaceutical preparations. A pharmaceutical preparation of a texaphyrin may be administered alone or in combination with pharmaceutically acceptable carriers, in either single or multiple doses. Suitable pharmaceutical carriers include inert solid diluents or fillers, sterile aqueous solution and various organic solvents. The pharmaceutical compositions formed by combining a texaphyrin of the present invention and the pharmaceutically acceptable carriers (including infusion and perfusion fluids) are then easily administered in a variety of dosage forms such as injectable solutions.

For parenteral administration, solutions of the texaphyrin in sesame or peanut oil, aqueous propylene glycol, saline, or in sterile aqueous solution may be employed. Such aqueous solutions should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy use with a syringe exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, cyclodextrin derivatives, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars such as mannitol or dextrose or sodium chloride. A more preferable isotonic agent is a mannitol solution of about 2-8% concentration, and, most preferably, of about 5% concentration. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

Texaphyrins may be impregnated into a stent by diffusion, for example, or coated onto the stent such as in a gel form, for example, using procedures known to one of skill in the art in light of the present disclosure.

Detection of Macrophage-Mediated Disease

Macrophage-mediated disease may be detected or imaged using texaphyrin in addition to being treated using texaphyrin and a co-therapeutic agent. Imaging may occur prior to or after treatment. Imaging and treatment may occur using a single texaphyrin that is both a detectable texaphyrin and a texaphyrin active with a co-therapeutic agent in one of the treatment modalities provided herein. Alternatively, a detectable texaphyrin may be administered for imaging, and a second texaphyrin having activity with a co-therapeutic agent as provided herein may be administered for treatment. As will be known by those skilled in the art, adjustment may be required to modulate energy administration between imaging and therapeutic levels.

For example, for imaging macrophage rich disease locuses of a subject, such as restenotic tissue, atheromatous plaque, or unstable or friable plaque, a detectable texaphyrin is administered to the subject and the restenotic tissue, atheromatous plaque, or unstable or friable plaque is imaged. Detectable texaphyrins may be imaged in a number of ways, for example, fluorescent texaphyrins may be used for detection. The term “fluorescent”, as used herein, means that upon photoirradiation by light associated with the absorption profile of texaphyrin, light is emitted at a longer wavelength by the irradiated texaphyrin. All texaphyrins are fluorescent, albeit, to varying degrees, and texaphyrins complexed with Y(III), Lu(III), Gd(III), Dy(III), Eu(III), or Mn(III) are presently preferred as fluorescent texaphyrins, for example.

In addition to fluorescent detection, texaphyrins may be imaged by x-radiation, by Raman scattering, magnetometry (bioluminiscence) or optical coherence tomography; further, texaphyrins complexed with a paramagnetic metal cation may be used for magnetic resonance imaging. Preferred paramagnetic metal cations include Mn(II), Mn(III), Fe(III), or trivalent lanthanide metals other than La(III), Lu(III), and Pm(III). Presently, the more preferred paramagnetic metal is Mn(II), Mn(III), Dy(III), or Gd(III); most preferably, Gd(III). Any of various types of magnetic resonance imaging can be employed in the practice of the invention, including, for example, nuclear magnetic resonance (NMR), NMR spectroscopy, and electronic spin resonance (ESR). The preferred imaging technique is NMR.

Gamma particle detection may be used to image a texaphyrin complexed to a gamma-emitting metal. ⁵¹Chromium, ⁶⁸gallium, ⁹⁹technetium, or ¹¹¹lindium are preferred metals for complexing to texaphyrins for gamma particle scanning. Monochromatic X-ray photon sources may be used for imaging also.

The texaphyrin to be used in the detection methods of the invention will be administered in a pharmaceutically effective amount. By “pharmaceutically effective” is meant a dose that will provide an image for detection by any one of the imaging methods described herein. The specific dose will vary depending on the particular texaphyrin chosen, the dosing regimen to be followed, timing of administration, the tissue to be imaged, and the physical delivery system in which it is carried.

A sufficient amount of texaphyrin is administered to produce an observable fluorescent emission when excited by light, preferably light having a wavelength in the range of about 400-500 nm (the Soret band) or 650-800 nm (the Q band). Images are recorded by illuminating with light in the excitation wavelength range and detecting the amount of fluorescent light emitted at the emission wavelength of preferably about 730-770 nm. Such dose can be determined without undue experimentation by methods known in the art or as described herein.

A further embodiment of the invention is a method of treating macrophage-mediated disease of a subject in need thereof comprising administering a chemotherapeutic agent and a detectable texaphyrin to the subject, and imaging the subject. This technique treats the macrophage-mediated disease with the chemotherapeutic agent having enhanced activity in the presence of the detectable texaphyrin, and allows for the monitoring of the macrophage-mediated disease, such as location and size of atheroma, for example.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Texaphyrin Accumulation in Macrophage of Atheromatous Plaque

Confocal Fluorescence Microscopy: Confocal laser scanning was performed on plaque ladened aortas excised from two hypercholesterolemic NZW rabbits. One cholesterol-fed rabbit was injected with lutetium texaphyrin (LuT2BET) (10 μmol/kg) over 2 consecutive days. The rabbit was sacrificed 24 hours after the last injection, the aorta was harvested, cut open and washed extensively with cold isotonic saline. The other cholesterol fed rabbit served as a control (no LuT2BET). The aorta specimens were viewed with a Sarastro™ 2000 Upright CLSM (Molecular Dynamics, Sunnyvale, Calif.). The samples were excited with 488 nm light from an argon ion laser. A 660 nm cut-off filter was used to collect the fluorescence emission signal.

The intracellular biodistribution pattern of lutetium texaphyrin was assessed. In the atheromatous plaque-ladened aorta of the lutetium texaphyrin-treated rabbbit, punctate irregular fluorescent spots were distributed throughout the atheroma. No fluorescence was detected in the rabbit that did not receive lutetium texaphyrin, confirming that the fluorescence observed in the treated rabbit was not due to inherent endogenous fluorescence.

The pattern of punctate fluorescence spots in the PCI-0123 treated rabbit was consistent with that expected for macrophage distribution, demonstrating that texaphyrin localizes in macrophages.

Antibody Staining to Confirm Uptake: The uptake of texaphyrin in activated macrophages is confirmed by following the above-described procedure, obtaining cryosections, staining with RAM 11 (an antibody that binds with a cytoplasmic antigen in rabbit macrophages (DAKO)).

Other Texaphyrins: By substituting LuT2BET with other texaphyrins, macrophage localization is similarly demonstrated.

### EXAMPLE 2

### Texaphyrin Destruction of Macrophage of Restenotic Tissue

The present example demonstrates that texaphyrins destroy macrophage.

Bilateral iliac artery lesions were induced in 9 male New Zealand White rabbits (NZW) after being on a high cholesterol diet (1%, Dyets, Bethlehem, Pa.) for 3 weeks. Each rabbit was anesthetized using a mixture of ketamine (5 mg/kg) and xylazine (35 mg/kg). The right carotid artery was exposed, carefully excised and a 5 Fr sheath (USCI) inserted into the descending aorta under fluoroscopic guidance. A 3.0 mm angioplasty balloon was advanced into the right or left iliac artery and then inflated distal to the deep femoral artery three times at 8 atm for 30 seconds. Subsequently, the same procedure was repeated in the contralateral iliac artery. Animals were maintained on the high cholesterol diet.

Six weeks after the denudation, each rabbit was reanesthetized. The left carotid artery was exposed, carefully excised and a 6 Fr sheath (USCI) inserted into the descending aorta under fluoroscopic guidance. A local drug delivery balloon (3 mm, Scimed, Minneapolis, Minn.) was advanced to the left or right iliac artery and placed at the same position as the previous balloon injury site. The proximal end of the delivery catheter was placed at the internal iliac artery under fluoroscopic control as a landmark reference. The balloon was inflated to 6 atm and 1 μmol/kg lutetium texaphyrin (LuT2BET) (2 mM in 5% mannitol) was infused intraarterially adjacent to the atheroma at a rate of 0.2 ml/min. Fifteen minutes post infusion a 0.9 mm optical fiber with a 3 cm cylindrical diffuser (Laserscope Inc., San Jose, Calif.) connected to an argon pumped dye laser (732 nm, Coherent Inc., Palo Alto, Calif.) delivered intravascular light at 180 J/cmF. One rabbit served as a light alone (no LuT2BET) control.

Two weeks following light treatment, each animal was euthanized and both iliac arteries were carefully removed from the adjacent tissue. Care was taken to harvest the exact portion of the artery where local drug delivery occurred by matching the anatomy with the respective fluoroscopic picture. The contralateral artery in the LuT2BET-treated rabbit served as a sensitizer alone control vessel. The vessels were fixed in 10% buffered formalin, embedded in paraffin and stained sequentially with RAM 11 (an antibody that binds with a cytoplasmic antigen in rabbit macrophages (DAKO)), and hematoxylin and eosin (H&E) for light microscopic analysis and planimetry.

Mean % plaque area in the treated segments was significantly smaller than in the nontreated segments (73±10% vs 81±12%, P<<0.01). Quantitative analysis using RAM 11 revealed significant reduction of macrophages in treated lesion in intima (5.1±5.3% versus 21±18%, P<<0.01) and in media (7.7±10% versus 22±20%, P<<0.01).

These results demonstrate that LuT2BET selectively destroys activated macrophages. By substituting LuT2BET with other texaphyrins, activated macrophage destruction is similarly demonstrated.

It is, therefore, concluded that texaphyrin plus a co-therapeutic agent, in this case, photoirradiation, is effective in treating macrophage-mediated disease, including reducing plaque following angioplasty and treating restenosis.

### EXAMPLE 3

### Hematology Study for Texaphyrin and Doxorubicin

The present example provides a summary of results obtained from a hematology study carried out on normal mice to test for any combined toxicity from gadolinium texaphyrin and doxorubicin.

A control group of eight Balb/c mice received no treatment. A second group of eight received injections of doxorubicin at 7.5 mg/kg/week for three weeks. A third group received injections of doxorubicin as group #2, followed 5 min later by GdT2BET at 40 μmol/kg/week for three weeks. Normal values were obtained from the California Veterinary Diagnostics, Inc. (West Sacramento, Calif.). White blood cell counts, red blood cell counts, hemoglobin values in gm/dL and platelet counts were obtained two weeks after the first injection and two weeks after the last injection.

Results clearly show no enhanced doxorubicin-induced bone marrow toxicity when the texaphyrin was used with doxorubicin, as measured by peripheral white blood cell count, platelet count and hemoglobin. In all four parameters studied, and in both time frames, values for the group of mice receiving doxorubicin and texaphyrin were very close to and within the error values found for the group of mice receiving doxorubicin only. These results emphasize the nontoxicity of texaphyrins in vivo, especially a lack of toxicity on bone marrow.

### EXAMPLE 4

### Cardiac Toxicity Study for Texaphyrin and Doxorubicin

The present example provides data that demonstrate that cardiac toxicity of doxorubicin is not exacerbated by the administration of gadolinium-texaphyrin.

One of the dose-limiting toxicities associated with doxorubicin (adriamycin) chemotherapy is cardiac toxicity. To test whether gadolinium texaphyrin exacerbates cardiac toxicity, Sprague Dawley rats were injected intravenously either with 5% mannitol (10 ml/kg, n=6), or doxorubicin (1 mg/kg, n=14), or doxorubicin (1 mg/kg) followed 1 hour later by gadolinium texaphyrin (20 micromoles/kg, n=14). The dosing schedule was repeated once a week for twelve weeks. Animals were sacrificed one week post the last dose, and hearts were fixed in formalin. Histologic evaluation revealed that doxorubicin in combination with gadolinium texaphyrin did not significantly exacerbate cardiac toxicity in comparison to those animals that received doxorubicin alone.

### EXAMPLE 5

### Radiation Therapy of Balloon-Injured Rat Carotid Artery in Preventing Restenosis

The present example provides studies on the efficacy of radiation therapy in the prevention of restenosis using GdT2BET as a radiation sensitizer.

The rat carotid model was used for the studies. Sprague-Dawley male rats weighing about 400-450 g were anesthetized and maintained with Ketamine/Rompun cocktail via the animal's tail vein. The rats were shaved to expose the chest and neck, and a midline incision was made to expose the left common carotid artery, the external carotid artery, and the internal carotid artery. The internal carotid artery was temporally ligated. A small incision was made in the external carotid artery and a 2F Fogarty arterial embolectomy balloon catheter (Baxter Cardiovascular Group, Irvine, Calif.) was introduced from the external carotid artery to the common carotid artery. The balloon was inflated with 0.2 ml of saline and withdrawn from near to the common carotid bifurcation with a rotating motion to produce uniform endothelial denudation and medial stretching in the common carotid artery. After 3 passages, the catheter was removed, the external carotid artery was ligated, and the internal carotid artery was untied. The incision was then closed.

GdT2BET (2mM in 5% mannitol) at 20 μmol/kg was injected via the tail vein 22 hours following balloon denudation of the carotid artery. Radiation therapy was performed 2 hours after the gadolinium texaphyrin injection. The treatment area was marked and a 250 kV X-ray radiation machine was used. The animals were anesthetized and maintained with Ketamine/Rompun cocktail and a special rat carotid artery jig was used for the treatment. The rat was strapped to a board under the X-ray head. A wooden frame was placed around the rat and lead shields were used to shield all body parts except a 1 inch slot around the common carotid artery area, such that only the treatment area was exposed to the X-ray. The radiation dose was 7 Gray.

A control group included three rats with balloon catheter injury only, a further control group included two rats with the balloon catheter injury and radiation treatment only, i.e., no gadolinium texaphyrin injection. The study group included four rats with the balloon catheter injury, gadolinium texaphyrin injection, and radiation treatment.

Animals were sacrificed 2 weeks post-treatment. The carotid artery was excised and fixed in buffered formalin. Haemotoxylin and eosin (H&E) stained sections were obtained. Full restenosis covering 100% of the arterial lumen was observed in all of the animals of each control group. Of the four animals that received gadolinium texaphyrin and radiation treatment, three had no evidence of restenosis, i.e., the vessels were observed as normal carotid arteries. The fourth animal had mild restenosis affecting up to 40% of the lumen circumference.

### EXAMPLE 6

### Determination of Antiinflammatory Activity Using Photodynamic Synovectomy Antigen-Induced Arthritis Model

An adaptation of the photodynamic synovectomy in an antigen-induced arthritis model as reported by Trauner, K., et al., (Lasers in Surgery and Medicine, 22:147-156 (1998)) is used to demonstrate antiinflammatory activity by texaphyrin.

Antigen-Induced Arthritis Model: New Zealand White rabbits are sensitized over a 6-week period with two cutaneous injections of ovalbumin (albumin, chicken egg, grade IV, Sigma, St. Louis, Mo.) suspended in Freunds adjuvant (1 ml of 10 mg/ml) (Sigma). Six weeks later, all knee joints are challenged with an intra-articular injection of 2.5 mg ovalbumin in 0.25 ml sterile saline solution. A monoarticular synovitis is then produced in 3-5 days.

Texaphyrin Administration: One week after joint challenge, experimental animals receive systemic intravenous injections of texaphyrin (2mM in 5% mannitol) at 1 μmol/kg. Control animals receive no texaphyrin.

Photodynamic Therapy: Two days post-texaphyrin injection, the right knee of each texaphyrin-exposed, anesthetized, animal receives photoirradiation at a wavelength of 732 nm via optical fibers through angiocatheters placed into the anteromedial and anterolateral compartments of the right knees. A total light energy of about 100 J/cm² is delivered to each joint at a power of 250 mW for 20 minutes. The left knees serve as controls receiving angiocatheters without optical fibers or light energy delivery.

The test animals are divided into two groups and sacrificed, respectively at two and four weeks post photodynamic therapy. The knee joints are dissected, the patellar tendon is transected, and the patella reflected laterally. The joint is examined grossly and a sample of synovium is retrieved from the infrapatellar fat pad and place in 10% neutral-buffered formalin for one week. The synovium is processed for glycol methacrylate embedment, cut into 3-5 μm sections and stained with H&E. The remaining knee joint is placed in 10% neutral-buffered formalin 3-4 weeks, split sagitally, and decalcified in 10% formic acid in sodium citrate buffer for 6-8 weeks. The medial and lateral specimens are embedded in paraffin and 5-7-μm-thick serial sections are cut and stained with H&E or Safranin O for proteoglycan content.

Samples are assessed for synovial necrosis, synovial thickness, plasma cell infiltrate, macrophage infiltrate, lymphocytic infiltrate, neutrophil infiltrate, cartilage integrity, appearance of bone, and appearance of articular cartilage. Statistical analysis of the ranked data for right and left knees of both control and treatment groups is performed for each parameter. Right and left knee scores are compared in control and treated animals. An improvement in assessed parameters for treated knees as compared to control knees indicates that texaphyrin is effective in photochemical synovectomy for patients suffering from rheumatoid arthritis.

When tested as described above, texaphyrin is effective in treating arthritis.

All of the methods disclosed and claimed herein can be executed without undue experimentation in light of the present disclosure. While the methods of this invention have been described in terms of preferred embodiments, it will be apparent to those of skill in the art that variations may be applied to the methods and in the steps or in the sequence of steps of the method described herein without departing from the concept, spirit and scope of the invention. All such variations apparent to those skilled in the art in light of the present disclosure are deemed to be within the spirit, scope and concept of the invention as defined by the appended claims.

1. Use of a texaphyrin in the preparation of a pharmaceutical composition for use in treating a macrophage-mediated disease of a subject in need thereof by sensitizing the effects of a co-therapeutic agent in macrophages of the subject, the composition comprising an effective amount of the texaphyrin and a pharmaceutically acceptable carrier.

2. The use of claim 1 wherein the co-therapeutic agent is a chemotherapeutic drug, light, radiation, or sonic energy.

3. The use of claim 1 wherein the macrophage-mediated disease is a cardiovascular disease, an autoimmune disease, a granulomatous disease, an inflammatory disease, or transplant.

4. The use of claim 1 wherein the texaphyrin is GdT2BET or LuT2BET.

5. The use of claim 1 wherein the macrophage-mediated disease is restenosis.

6. The use of claim 1 wherein the macrophage-mediated disease is atheroma and the co-therapeutic agent is a chemotherapeutic drug, radiation or sonic energy.

7. Use of a texaphyrin in the preparation of a pharmaceutical composition for use in imaging and treating macrophage-mediated disease of a subject in need thereof by sensitizing the effects of a co-therapeutic agent in macrophages of the subject, the composition comprising an effective amount of the texaphyrin and a pharmaceutically acceptable carrier.

8. The use of claim 7 wherein the co-therapeutic agent is a chemotherapeutic drug, light, radiation, or sonic energy.

9. The use of claim 7 wherein the macrophage-mediated disease is a cardiovascular disease, an autoimmune disease, a granulomatous disease, an inflammatory disease, or transplant.

10. The use of claim 7 wherein the texaphyrin is GdT2BET or LuT2BET.

11. The use of claim 7 wherein the macrophage-mediated disease is restenosis.

12. The use of claim 7 wherein the macrophage-mediated disease is atheroma and the co-therapeutic agent is a chemotherapeutic drug, radiation or sonic energy.

13. The use of claim 7 wherein the imaging is by detecting fluorescence of the texaphyrin.

14. The use of claim 7 wherein the texaphyrin is complexed with a paramagnetic metal and the imaging is by magnetic resonance imaging.

15. A method of treating a macrophage-mediated disease of a subject in need thereof by sensitizing the effects of a co-therapeutic agent in macrophages of the subject comprising administering an effective amount of a texaphyrin and an effective amount of the co-therapeutic agent to the subject.

16. The method of claim 15 wherein the co-therapeutic agent is a chemotherapeutic drug, light, radiation, or sonic energy.

17. The method of claim 15 wherein the macrophage-mediated disease is a cardiovascular disease, an autoimmune disease, a granulomatous disease, an inflammatory disease, or transplant.

18. The method of claim 15 wherein the texaphyrin is GdT2BET or LuT2BET.

19. The method of claim 15 wherein the macrophage-mediated disease is restenosis.

20. The method of claim 15 wherein the macrophage-mediated disease is atheroma and the co-therapeutic agent is a chemotherapeutic drug, radiation or sonic energy.

21. A method of imaging and treating macrophage-mediated disease of a subject in need thereof comprising
administering an effective amount of a texaphyrin to the subject;
imaging the subject; and
administering an effective amount of a co-therapeutic agent to the subject.

22. The method of claim 21 wherein co-therapeutic agent is a chemotherapeutic drug, light, radiation, or sonic energy.

23. The method of claim 21 wherein the macrophage-mediated disease is a cardiovascular disease, an autoimmune disease, a granulomatous disease, an inflammatory disease, or transplant.

24. The method of claim 21 wherein the texaphyrin is GdT2BET or LuT2BET.

25. The method of claim 21 wherein the macrophage-mediated disease is restenosis.

26. The method of claim 21 wherein the macrophage-mediated disease is atheroma and the co-therapeutic agent is a chemotherapeutic drug, radiation or sonic energy.

27. The method of claim 21 wherein the imaging is by detecting fluorescence of the texaphyrin.

28. The method of claim 21 wherein the texaphyrin is complexed with a paramagnetic metal and the imaging is by magnetic resonance imaging.
